(19) United States
(12) Patent Application Publication
(10) Pub. No.: US 2010/0228267 A1
(43) Pub. Date: Sep. 9, 2010

(54) Wound care instrument

(76) Inventor: Axel Luis Mercado, Miami, FL (US)

Correspondence Address: Axel Luis Mercado, 7041 Torphin PL, Miami Lakes, FL, 33014, US

(21) Appl. No.: 12/661,420

(22) Filed: Mar. 4, 2010

Related U.S. Application Data
(60) Provisional application No. 61/157,953, filed on Mar. 6, 2009.

(51) Int. Cl. A61B 17/50

(52) U.S. Cl. 606/131

(57) ABSTRACT

A wound care instrument to be used for measuring, probing, suctioning, cleaning, mechanically debriding, and applying creams or ointments in or onto acute and chronic ulcers or wounds, including but not limited to undermining or tunneling, surgically dehisced wounds, penetrating wounds, and burns.

This application addendum is based on a “Notice To File Missing Parts of Non-Provisional Application”, under Application Ser. No. 12/661,420 with original filing of File No: 1341.AHC10003P (Ser. No. 61/157,953) file on Mar. 6, 2009.

### BACKGROUND OF THE INVENTION

Current ulcer and wound care procedures and instruments fail to adequately address the need for proper ulcer and wound bed preparation by decreasing microbial colonization in the wound or ulcer other than using a medicated dressing or cream or ointment. They also fail to address the need for adequate removal of drainage in a wound or ulcer site that has become infected as a result of an infectious process, or the creation of a nidus for infection due to the presence of residual cleaning solution that was not properly evacuated from the wound or ulcer site during the cleaning of the ulcer or wound.

By addressing the problems associated with microbial colonization and infection prevention in pressure ulcers, surgically dehisced wounds, open wounds, or burns, a practitioner can substantially improve the bed state of an ulcer or wound by promoting a healing environment while significantly reducing the cost of treating these ulcers or wounds.

It is therefore an object of the present invention to provide a wound care instrument to assist practitioners in cleaning of ulcers, open wounds, and burns in the delivery of ulcer or wound care. It is another object of the present invention to reduce the rate of ulcers, wounds, and burns from worsening and advancing in stage or deteriorating due to increased fibrotic and necrotic tissue thus leading to excisional debridements. It is another object of the present invention to provide a wound care instrument that will help improve healing rates for patient's receiving ulcer or wound care in hospitals, long-term acute care facilities (LTACs), skilled nursing facilities (SNFs), nursing homes (NHs), Adult Living Facilities (ALF's), out-patient offices, wound care centers (WCCs), or patient's homes. It is yet another object of the present invention to provide a wound care instrument that will help substantially decrease the costs associated with wound or ulcer care and therapy.

### SUMMARY OF THE INVENTION

The present invention is generally described as a wound care instrument to be used for measuring, probing, suctioning, cleaning, mechanically debriding, and applying creams or ointments in or onto acute and chronic (pressure, venous, arterial, diabetic) ulcers or wounds, with undermining or tunneling, surgically dehisced wounds, penetrating wounds, and burns.

Preferably, the present invention is comprised of a tube which is between approximately 10 and 20 cm in length, is hollow, and has a diameter of approximately 5 to 6 mm. Preferably, the tube is made out of plastic. Preferably, the tube can be connected to a suctioning pump or similar device through a yank cord attachment that can be permanently affixed or selectively detachable to the tube. Preferably, the tube has markings on at least one surface for measuring the surface, depth and any undermining or tunneling at the ulcer or wound site. These markings will preferably start on the end proximal to the suctioning device.

The present invention is further comprised of a first tip with an opening on each end and preferably made of an abrasive material for debriding wounds or ulcers. This first tip can be attached onto the end of the tube distal to the suctioning device. In this embodiment, the present invention can be used to suction and mechanically debride ulcer or wounds. The present invention is further comprised of a second tip preferably having a single opening which can also be attached to the end of the tube distal to the suctioning device. In this embodiment of the present invention can be used to apply creams or ointments onto or into ulcers or wound beds. The first and second tip can be permanently affixed to the tube, or alternatively, selectively detachable to the tube. The tube, first tip, second tip, and yank cord can also be individually accessible to the user, or collectively as a kit.

The present invention diminishes ulcer or wound colonization and reduces the risk of infections by effectively suctioning out drainage, purulent discharge, and residual cleansing solutions from the ulcer or wound site. The present invention also assists with physical mechanical debridement of the ulcer or wound bed, thus minimizing the likelihood of ulcer or wound colonization by removing slough, necrotic tissue, and excess drainage or exudates which will be suctioned out during the mechanical debridement process. The present invention also assists in providing a cleaner ulcer or wound environment therefore enhancing the healing process. In addition, the present invention will be able to measure size, depth, and undermining or tunneling of an ulcer or wound site.

Another embodiment of the present invention is generally comprised of a shaft preferably measuring between approximately 15 and 20 cm in length, having a flat surface that is tapered on at least one end, and a diameter of approximately 4 to 6 mm. This embodiment is preferably made out of a transparent plastic, and has markings used for measuring the surface, depth and any undermining or tunneling at the wound or ulcer site. Preferably these markings are etched or printed onto the surface of the shaft and will begin on the tapered end of the shaft.

This embodiment of the present invention is further comprised of at least one end having an abrasive surface on one side of the shaft and an applicator surface on the other side of the shaft used for mechanically debriding ulcers or wounds and also applying creams or ointments to the site.

This particular embodiment of the present invention can assist with physical mechanical debridement of the ulcer or wound bed to also minimize the likelihood of ulcer or wound colonization by removing slough and necrotic tissue, or clean a stagnant ulcer or wound bed. This embodiment of the present invention can also assist in providing a cleaner ulcer or wound environment, thereby enhancing the healing process. In addition, this embodiment of the present invention can measure the size, depth, and undermining or tunneling in a wound or ulcer site.

### BRIEF DESCRIPTION OF DRAWINGS

FIG. 1 represents a view of the top of the suctioning instrument with the distal end of the instrument connected to rubber tubing attached onto connectors for suctioning apparatuses.

FIG. 2 represents a view of the top of the suctioning instrument with the proximal distal end of the instrument attached to device to be used for performing non-excisional maintenance mechanical debridements.

FIG. 3 represents a view of the top of the suctioning instrument with the proximal tip of the instrument attached to device to be used for applying creams and ointments.

FIG. 4 represents a view of the top of the non-suctioning version of the instrument with the distal end of the instrument with a abrasive surface for performing non-excisional maintenance mechanical debridements.

FIG. 5 represents a view of the side demonstrating the distal top end of the non-suctioning version of the instrument with an abrasive surface and having the bottom distal end of the instrument with a non-abrasive surface to be used for applying creams and ointments.

### DETAILED DESCRIPTION OF THE PREFERRED EMBODIMENTS

The present invention is generally described as a wound care instrument to be used for measuring, probing, suctioning, cleaning, mechanically debriding, and applying creams or ointments in or onto acute and chronic (pressure, venous, arterial, diabetic) ulcers or wounds, including but not limited to undermining or tunneling, surgically dehisced wounds, penetrating wounds, and burns. The present invention can be sterile or non-sterile. The present invention can be re-useable or disposable.

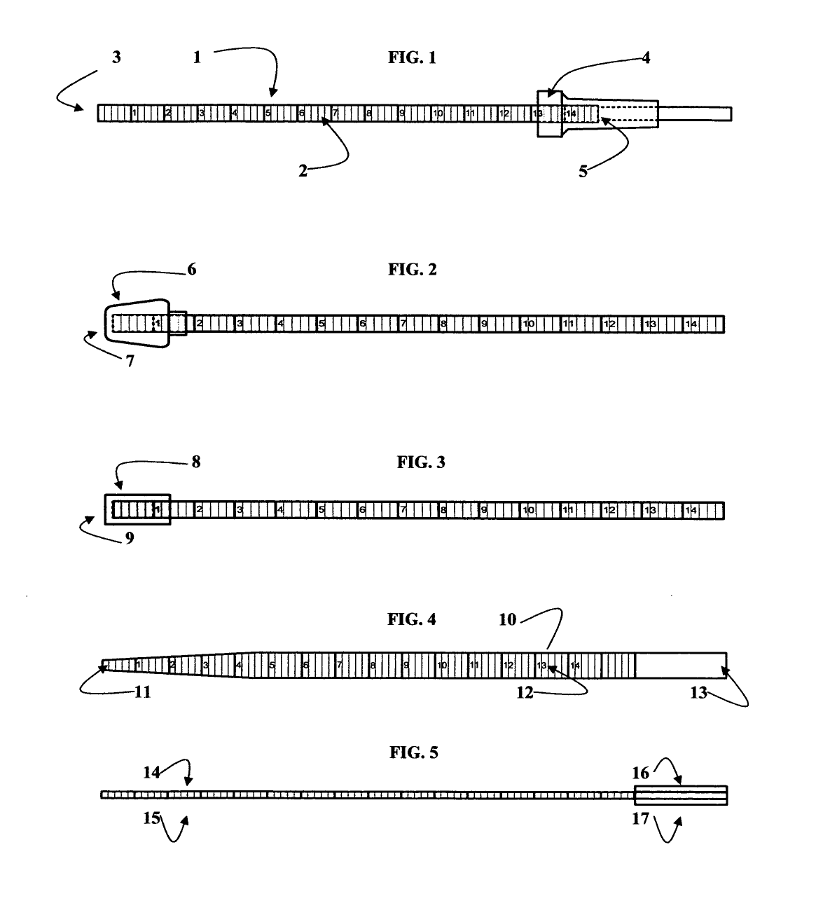

As illustrated in FIG. 1, in one embodiment of the present invention, the tube 1 is between approximately 10 to 25 cm in length; is hollow; has openings on both ends; and has a diameter of approximately 3 to 7 mm. Preferably, the length of tube 1 is between 15 and 20 cm in length, and has a diameter approximately between 5 and 6 mm. The tube 1 is preferably made out of a transparent plastic, but can be made of any material suitable for the purpose. The tube can also be opaque or colored. The cross-section of tube 1 is preferably circular, but can also be comprised of any geometric-shape, including but not limited to square-shaped, flat, beveled, oval-shaped, semi-lunar, rectangular, triangular, pentagonal, sectagonal, hexagonal, octagonal, etc.

The tube 1 is further comprised of a scale 2 used for measuring the depth or length of an ulcer or wound. This scale 2 can be etched or printed onto the outer or inner surface of the tube 1. Scale 2 can utilize any system of measurement suitable for the purpose, including but not limited to inches, centimeters, or millimeters. The scale 2 begins on the first end 3 of the tube 1. Alternatively, the scale begins on the second end 5 of the tube 1. The scale 2 can be etched or printed on one side of the tube 1. In another embodiment of the present invention, a second scale can be etched or printed on the other side of the tube 1. The scale 2 can be used to measure the surface, depth, and any undermining or tunneling at the site of the ulcer.

As further illustrated by FIG. 1, a yank cord 4 can be attached to the second end 5 of tube 1. Yank cord 4 connects tube 1 to a suctioning pump (or similar device) thereby allowing the first end 3 of the tube 1 to apply suction to a desired site. Yank cord 4 is preferably hollow, and has a first entrance that can sleeve the second end 5 of tube 1. Yank cord 4 preferably has a second entrance that can sleeve a conduit to a suctioning source, such as a length of tubing attached to a suction pump, or a syringe. Preferably, yank cord 4 is constructed of a semi-flexible or rigid plastic, but can also be constructed of any material suitable for the purpose.

As illustrated in FIG. 2, in another embodiment of the present invention, a debridement tip 6 is attached to the first end 3 of the tube 1. Preferably, debridement tip 6 is hollow; has an opening on each end, at least one of which permits the passage of tube 1; has a flat suctioning end 7; and is preferably comprised of an abrasive material, but can also be made of any material suitable for mechanically debriding ulcers or wounds. In another embodiment of the present invention, debridement tip can be further configured to receive a blade, or similar instrument, capable of assisting in mechanically debriding the ulcer or wound.

As illustrated in FIG. 3, in another embodiment of the present invention, an applicator tip 8 is attached to the first end 3 of the tube 1. Applicator tip 8 has a single opening at one end which permits the passage of tube 1; has a flat closed end 9; and is preferably made of a foam-like material, but can also be made of any material suitable for applying creams or ointments onto or into an ulcer or wound.

Debridement tip 6 and applicator tip 8 can be selectively and interchangeably attached to the first end of tube 1 by the user. In this particular embodiment, debridement tip 6, applicator tip 8, and tube 1 can be accessible to the user separately or as a kit. In an alternative embodiment of the present invention, debridement tip 6 can be permanently affixed to the first end of tube 1; and in yet another embodiment of the present invention, applicator tip 7 can be permanently affixed to the first end of tube 1. Yank cord 4 can also be selectively and interchangeably attached to the second end of tube 1; in this embodiment, yank cord 4, tube 1, debridement tip 6, and applicator tip 8 can be accessible to the user individually or as a kit. Alternatively, yank cord 4 can be permanently attached to the second end of tube 1.

As illustrated in FIGS. 4 and 5, in another embodiment of the present invention, the wound care instrument is comprised of a planar shaft 10 of approximately between 10 and 25 cm in length, and preferably between 15 and 20 cm in length; approximately between 0.1 and 2.0 cm in width, and preferably between 0.5 and 1.5 cm in width; and approximately between 0.1 and 1.5 mm in height, and preferably between 0.2 and 0.8 mm in height. Shaft 10 is preferably made out of a transparent plastic, but can also be made of any material suitable for the purpose. The tube can also be opaque or colored. As depicted in FIG. 4, shaft 10 is substantially rectangular when viewed from its top surface 14 or its bottom surface 15, and preferably tapers into a flat first end 11. As depicted in FIG. 5, shaft 10 is preferably substantially rectangular in shape when viewed laterally. Alternatively, shaft 10 can have any geometric configuration.

Shaft 10 is further comprised of a scale 12 used for measuring the depth or length of an ulcer or wound. Scale 12 can utilize any system of measurement suitable for the purpose, including but not limited to inches, centimeters, or millimeters. The scale 12 can be etched or printed onto the top surface 14 or bottom surface 15 of the shaft 10. Alternatively, scale 12 can be printed or etched on both the top surface 14 and bottom surface 15 of the shaft 10. In one embodiment of the present invention, the scale 12 begins on the flat first end 11 of the shaft 10. In another embodiment of the present invention, the scale 12 begins on the second end 13 of the shaft 10. The scale 12 can be used to measure the surface, depth, and any undermining or tunneling at the site of the ulcer.

The second end 13 of the shaft 10 is further comprised of an abrasive pad 16 that is adjacent to the top surface 14, and an applicator pad 17 that is adjacent to the bottom surface 15. Abrasive pad 16 is approximately between 0.5 and 1.5 cm in length, and preferably between 0.8 and 1.2 cm in length; approximately between 0.1 and 1.0 cm in width, and preferably between 0.3 and 0.8 cm in width; approximately between 0.05 and 0.15 mm in height, and preferably between 0.8 and 0.12 mm in height; and is preferably comprised of an abrasive material, but can also be made of any material suitable for mechanically debriding ulcers or wounds. Applicator pad 17 is is approximately between 0.5 and 1.5 cm in length, and preferably between 0.8 and 1.2 cm in length; approximately between 0.1 and 1.0 cm in width, and preferably between 0.3 and 0.8 cm in width; approximately between 0.05 and 0.15 mm in height, and preferably between 0.8 and 0.12 mm in height; and is preferably made of a foam-like material, but can also be made of any material suitable for applying creams or ointments onto or into an ulcer or wound.

1. An instrument used to treat ulcers or wounds, comprising:
a hollow tube having a first end and a second end;
a scale on at least one surface of said tube;

2. The instrument of claim 1 further comprising a yank cord attached to at least one end of said tube.

3. The instrument of claim 1 further comprising a debridement tip attached to at least one end of said tube.

4. The instrument of claim 1 further comprising an applicator tip attached to at least one end of said tube.

5. A kit used to treat ulcers or wounds comprising:
a tube having a first end, a second end, and a scale on at least one surface of said tube;
a yank cord attachable to at least one end of said tube;
a debridement tip attachable to at least one end of said tube; and
an applicator tip attachable to at least one end of said tube.

6. An instrument used to treat ulcers or wounds, comprising:
a shaft having a first end, a second end, a top surface, and a bottom surface;
a scale on at least one surface of said shaft;
an abrasive surface on at least one end and at least one surface of said shaft; and
an applicator surface on at least one end and at least one surface of said shaft.

7. The instrument of claim 6, said first end being tapered.
